(19) United States
(12) Patent Application Publication
(10) Pub. No.: US 2012/0158064 A1
(43) Pub. Date: Jun. 21, 2012

(54) CURVED SPINAL CROSS-CONNECTOR

(76) Inventor: Matthew Kroll, Oakland Twp, MI (US)

(21) Appl. No.: 12/975,159

(22) Filed: Dec. 21, 2010

(51) Int. Cl. A61B 17/70

(52) U.S. Cl. 606/250

(57) ABSTRACT

Disclosed is an implantable medical device, specifically a spinal cross-connector, for connection to approximately parallel spinal fixation rods, the spinal fixation rods being configured to attach to the vertebrae of the spine. The spinal cross-connector may be curved to facilitate installation of the cross-connector over the vertebrae of the spine and allow space for the muscles along the spinal column to heal. The spinal cross-connector may also provide three degrees of adjustment of the spinal cross-connector. The spinal cross-connector may be adjustable or telescoping to vary the width of the spinal cross-connector according to an individual's anatomy and the distance between the spinal fixation rods. The spinal cross-connector may also facilitate rotation of a longitudinal member around a vertical axis. The spinal cross-connector may also facilitate rotation of a longitudinal member around a longitudinal axis. Once installed, the spinal cross-connector protects against torsional forces acting on the spinal fixation rods which may be disruptive to fusion and/or healing of the vertebrae fixed to the spinal fixation rods.

### BACKGROUND

Spinal fixation systems are employed in the context of orthopedic surgery to stabilize vertebrae of the spine and to establish or correct the spatial relationship between separate vertebra. In some cases, two spinal fixation rods running in an approximately parallel manner are affixed to multiple vertebrae of the spine on opposite sides of the spinous process by fasteners such as pedicle screws. The spinal fixation rods act as anchoring devices, immobilizing the vertebrae in a desired spatial relationship in order to facilitate spinal fusion or healing.

A spinal cross-connector may be used in conjunction with the spinal fixation rods to provide torsional stiffness to the spinal fixation rods and to provide additional stability to the vertebrae in order to help facilitate spinal fusion or healing. Spinal cross-connectors are preferably adjustable in some fashion to accommodate the contours of the spinal fixation rods attached to the spine. Depending on the size of the spinal fixation, one or more cross-connectors may be utilized.

Linear or uncurved spinal cross-connectors have been used historically because of the ease of manufacture and adjustment, but often interfere with the spinal anatomy. Such interference may prove painful to the patient and may also prevent a secure attachment of the spinal cross-connector to the spinal fixation rods.

Employment of curved cross-connectors may be beneficial in avoiding and protecting the anatomy of the spine. However, implementing the desired adjustability of the cross-connector to conform with the positioning of the spinal fixation rods has proven difficult with curved cross-connectors. Typically the attempts at implementing curved cross-connectors have been overly complex and expensive. Furthermore, because of the difficulty in adjusting the curved cross-connectors found in previous attempts, a secure attachment to the spinal fixation rods has been difficult to establish.

### SUMMARY

One embodiment of the invention comprises a spinal cross-connector for attachment to spinal fixation rods, the cross-connector comprising a coupler comprising a head portion and a threaded portion, wherein the head portion comprises a bore therethrough and the threaded portion extends from the head portion. The embodiment may further comprise a first longitudinal member comprising a spinal fixation rod coupler on a first end and a curved arm extending from the first end and through the bore of the coupler and a second longitudinal member comprising a spinal fixation rod coupler on a first end and an annular flange at a second end, wherein the threaded portion of the coupler extends through the annular flange. The embodiment may further comprise a washer positioned over the threaded portion of the coupler between the annular flange of the second longitudinal member and the head portion of the coupler and a nut engaged with the threaded portion of the coupler and located above the annular flange of the second longitudinal member.

In some embodiments, the threaded portion of the coupler extends along an axis perpendicular to the bore of the coupler. In some embodiments, the bore in the head portion of the coupler is oblong in vertical cross section. In some embodiments, the curved arm of the first longitudinal member may contact the bore at only two contact locations.

In some embodiments, the washer may contact portions of the curved arm at two locations opposite to the contact points between the bore and the curved arm. In some embodiments, the nut is tightened against a top surface of the annular flange and the washer is forced against the top portion of the curved arm.

In some embodiments, the curved arm of the first longitudinal member includes two straight sides and a rounded top and a rounded bottom. In some embodiments, the washer may include two skirt portions, which may be configured to fit against the straight sides of the curved arm of the first longitudinal member.

Some embodiments comprise a pin extending through a distal bore in the distal end of the curved arm, the distal bore running in a direction transverse to the bore in the coupler, wherein the pin is of a length greater than the smallest cross-sectional diameter of the bore in the coupler. Some embodiments may comprise two set screws, each set screw extending through a threaded bore in the first and second longitudinal members. Each of the screws engages a spinal fixation rod and secures the spinal fixation rod to the first and second longitudinal members.

In some embodiments, the curved arm of the first longitudinal member is configured to move longitudinally through the bore in the coupler. The curved arm of the first longitudinal member may also be rotatable inside the bore of the coupler. The annular flange of the second longitudinal member may be configured to rotate radially around the threaded portion of the coupler.

In another embodiment, a spinal cross-connector for attachment to spinal fixation rods comprises a first curved longitudinal member, a second longitudinal member, a coupler for coupling the first and second longitudinal members, wherein the coupler comprises an oblong bore configured to accommodate the curve of the first longitudinal member, wherein the first longitudinal member is rotatable around a longitudinal axis and is configured to move along the longitudinal axis through the coupler, and wherein the second longitudinal member is rotatable around a vertical axis.

In another embodiment, a spinal cross-connector for attachment to spinal fixation rods comprises a first curved longitudinal member, a second longitudinal member, a coupler for coupling the first and second longitudinal members, wherein the coupler comprises an oblong bore configured to accommodate the curve of the first longitudinal member, wherein the first longitudinal member is configured to move along a longitudinal axis through the coupler, and wherein the second longitudinal member is rotatable around a longitudinal and a vertical axis.

In another embodiment, a spinal cross-connector for attachment to spinal fixation rods comprises a first longitudinal member, a second longitudinal member, and means for coupling the first and second longitudinal members, wherein the coupling means allows the first longitudinal member to move laterally along the longitudinal axis through the coupling means and allows the first and second longitudinal member to rotate relative to one another around a vertical axis and to rotate relatively around a longitudinal axis. The means for coupling may be a coupler comprising a head portion and a threaded portion, wherein the head portion comprises a bore therethrough and the threaded portion extends from the head portion.

### BRIEF DESCRIPTION OF THE DRAWINGS

FIG. 1 provides a prospective view of the disassembled components which comprise an embodiment of the spinal cross-connector.

FIG. 2 is a side view of an embodiment of the assembled spinal cross-connector.

FIG. 3 is a top view of an embodiment of the assembled spinal cross-connector.

FIG. 4 is a cross-sectional side view of an embodiment of the spinal cross-connector corresponding to FIG. 2.

FIG. 5 is a perspective view of an embodiment of the coupler.

FIG. 6 is another cross-sectional view of an embodiment of the spinal cross-connector.

FIG. 7 is a perspective view of several assembled components of an embodiment of the spinal cross-connector.

FIG. 8 provides a perspective view of the disassembled components which comprise an embodiment of the spinal cross-connector.

FIG. 9 is a side view of an embodiment of the assembled spinal cross-connector.

FIG. 10 is a cross-sectional view of an embodiment of the spinal cross-connector.

### DETAILED DESCRIPTION

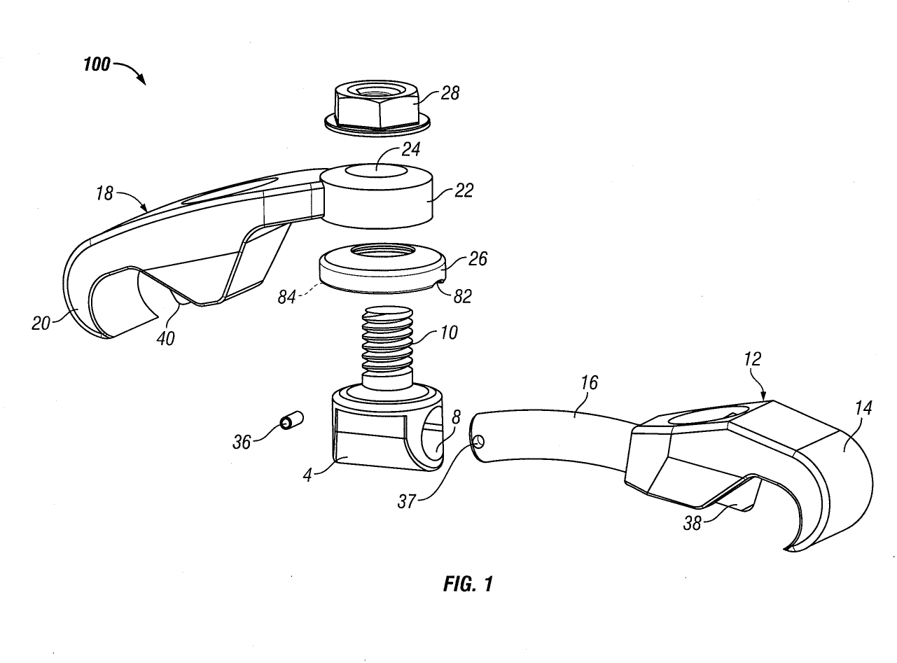

FIGS. 1-10 depict a spinal cross-connector, embodiments of which will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments disclosed herein. Furthermore, embodiments disclosed herein may include several novel features, no single one of which is solely responsible for its desirable attributes or which is essential to the embodiments herein described.

The technology described herein provides an implantable medical device, specifically a spinal cross-connector, for connection to approximately parallel spinal fixation rods. The spinal fixation rods are configured to attach to the vertebrae of the spine.

The spinal cross-connector disclosed herein is curved to facilitate installation of the cross-connector over the vertebrae of the spine and allow space for the muscles along the spinal column. Historically, implementing the desired adjustability to conform with the positioning of the spinal fixation rods has proven difficult with curved cross-connectors and typically the attempts have been overly complex, expensive to implement, and have had difficulty in establishing a secure connection with the spinal fixation rods.

The curved spinal cross-connector disclosed herein maintains the distinct advantages of a curved spinal cross-connector, but is also extremely easy to implement and adjust, and likewise provides a secure connection to the spinal fixation rods. As seen in FIG. 1, some implementations of the spinal cross-connector 100 comprise eight components: a first longitudinal member 12, a second longitudinal member 18, a coupler 4, a washer 26, a pin 36, and a nut 28. Housed within the first and second longitudinal members 12, 18 are respective set screws 38, 40. As will be explained further below, the washer 26 may be provided with cutouts 82, 84 on opposed lower portions thereof, only one of which is visible in FIG. 1. The components of the spinal cross-connector 100 may be made of medical-grade stainless steel, titanium, or other such material suitable for placement in the human body.

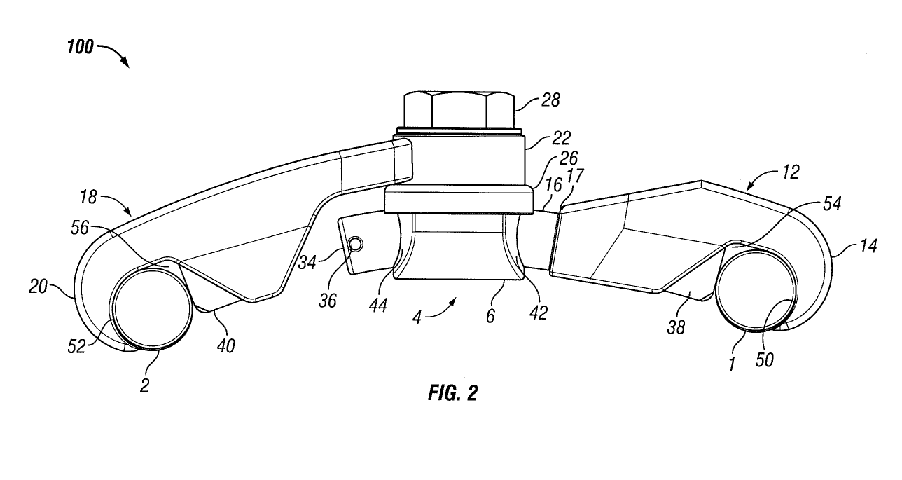

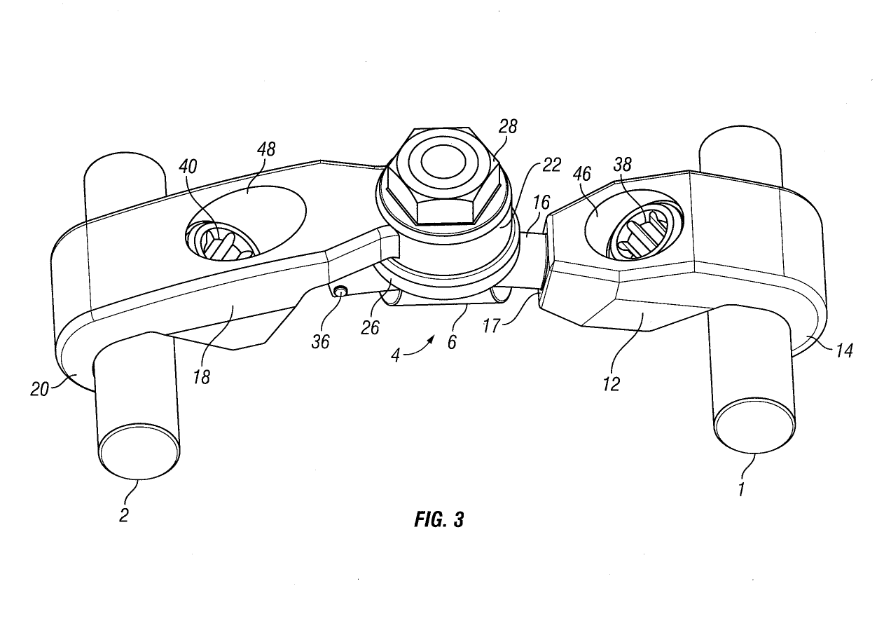

FIG. 2 provides a side view of the assembled spinal cross-connector 100 as attached to spinal fixation rods 1, 2. FIG. 3 is a perspective view from above the spinal cross-connector 100. Like FIG. 1, FIGS. 2 and 3 show the cross-connector 100 comprises eight components: the first longitudinal member 12, the second longitudinal member 18, the coupler 4, the washer 26, the pin 36, and the nut 28. Also visible in FIGS. 2 and 3 are the set screws 38, 40.

The first longitudinal member 12 comprises a spinal fixation rod coupler 14 on a first end and a curved arm 16 on a second end. When the spinal cross-connector 100 is assembled, the curved arm 16 extends through the bore 8 in the coupler 4 from a first side 42 of the coupler 4 and exits the bore 8 from a second side 44 of the coupler 4. The curved arm 16 has a distal end 34, the distal end 34 comprising a bore 37 running in a direction generally transverse to the length of the first longitudinal member 12. The bore 37 in the first longitudinal member 12 houses the pin 36 extending through it. The pin 36 is of a length greater than the smallest cross-sectional diameter of the bore 8 in the coupler 4, such that when the curved arm 16 is positioned in the coupler 4, the pin 36 prevents the distal end 34 of the curved arm 16 from passing back through the bore 8 in the coupler 4.

The second longitudinal member 18 comprises a spinal fixation rod coupler 20 on a first end and an annular flange 22 on a second end. The annular flange 22 comprises a bore 24 therethrough, which, when the spinal cross-connector is assembled, is positioned around a threaded portion 10 of the coupler 4. The washer 26 is also positioned around the threaded portion 10 of the coupler 4 and is disposed between the coupler 4 and the annular flange 22 of the second longitudinal member 18. The nut 28 can be tightened on the threaded portion 10 of the coupler 4 above the annular flange 22 of the second longitudinal member 18 to secure the component pieces of the spinal cross-connector 100 in the desired spatial arrangement.

Set screw 38 extends through a bore 46 in the first longitudinal member 12 into a rod receiving space 54. In the rod receiving space 54, the set screw 38 contacts and engages the spinal fixation rod 1. The set screw 40 extends through a bore 48 in the second longitudinal member 18 into a rod receiving space 56. In the rod receiving space 56, the set screw 40 contacts and engages the spinal fixation rod 2. Set screws 38, 40 engage and hold the spinal fixation rods 1, 2 in place against interior surfaces 50, 52 of the spinal fixation rod couplers 14, 20.

In addition to being curved, the first longitudinal member 12 can slide and rotate or twist within the bore 8 and the second longitudinal member 18 can rotate on the threaded portion 10 of the coupler 4. Each of these adjustments may be utilized individually or in conjunction with one another depending on the positioning of the spinal fixation rods to which the spinal cross-connector is to be attached and depending on the anatomy of the patient.

The rotation/twisting of the first longitudinal member 12 within the bore 8 of the coupler 4 is possible, despite the curvature of the curved arm 16, because the bore 8 is oblong shaped. The bore 8 has a sufficiently large vertical diameter such that the curvature of the curved arm 16 is accommodated in the oblong-shaped bore 8.

The first longitudinal member 12 can slide longitudinally back and forth through the bore 8 in the coupler 4 to adjust the width of the spinal cross-connector 100. The spinal cross-connector 100 is longitudinally adjustable to vary the width of the spinal cross-connector 100 according to an individual's anatomy and the distance between the spinal fixation rods.

The spinal cross-connector 100 also facilitates rotation of the second longitudinal member 18 around a vertical axis. When the second longitudinal member 18 is placed over the threaded portion 10 of the coupler 4, the second longitudinal member is rotatable around the vertical axis of the threaded portion 10 of the coupler 4 until the nut 28 is securely tightened down.

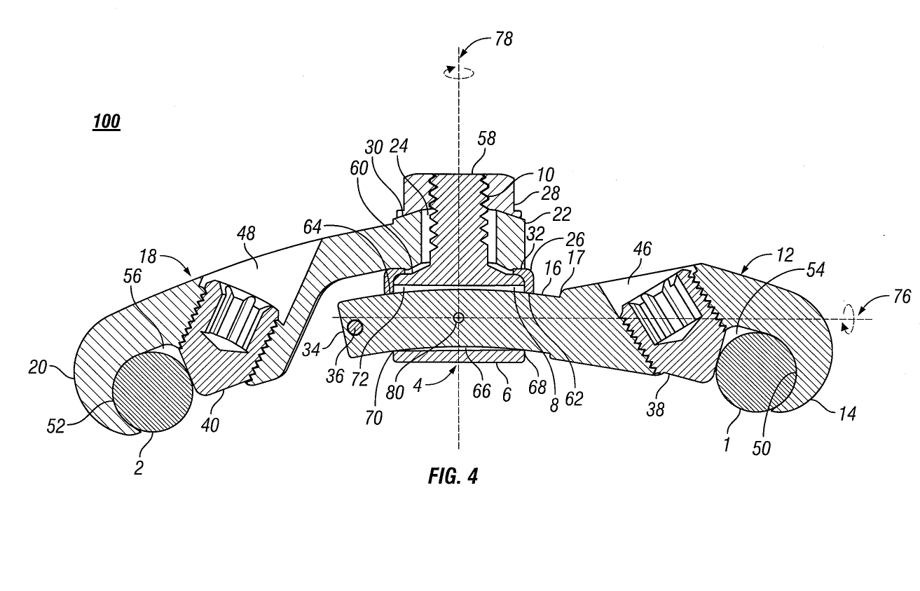

FIG. 4 shows a cross-sectional view of the spinal cross-connector 100 in an assembled state. Seen in FIG. 4 is the coupler 4 which comprises a head portion 6 and a threaded portion 10. The head portion 6 has a bore 8 and the threaded portion 10 extends from the head portion 6. Also visible in FIG. 4 are the bores 46, 48 through which the set screws 38, 40 extend through the first and second longitudinal members 12, 18 into the rod receiving spaces 54, 56. Also visible in FIG. 4 is the bore 24 of the annular flange 22 of the second longitudinal member 18. The bores 46, 48 are threaded for enabling the set screws 38, 40 to be tightened within the bores 46, 48 in applying pressure against and securing the spinal fixation rods 1, 2. The bores 46, 48 may be slanted outwards towards the rod receiving spaces 54, 56 in order to better allow the set screws 38, 40 to engage the spinal fixation rods 1, 2 against the interior surfaces 50, 52 of the spinal fixation rod couplers 14, 20.

The curved arm 16 of the first longitudinal member 12 extends through the bore 8 in the coupler. Because of the curvature of the curved arm 16, the curved arm 16 touches the bottom of the bore 8 of the coupler 4 at two contact points 68, 70, creating a lower gap 66 between the curved arm 16 and the bottom of the bore 8. The bore 8 in the coupler 4 has a sufficiently large vertical diameter that as the curved arm 16 contacts the coupler 4 at contact points 68, 70 on the bottom of the bore 8, an upper gap 72 is created in the bore 8 between the top of the curved arm 16 and the top of the bore 8. Thus, when the curved arm 16 is in place in the bore 8, the curved arm 16 does not contact the upper side of the bore 8. As described further below, the existence of the upper gap 70 and lower gap 66 allows for the rotation/twisting of the curved arm 16 within the bore 8.

Also seen in the cross-sectional view of FIG. 4 is the nut 28 located above the annular flange 22 of the second longitudinal member 18. The nut may contact the top surface 30 of the annular flange 22. A washer 26 is also disposed around the threaded portion 10 of the coupler 4. As the nut 28 is tightened on the threaded portion 10 of the coupler 4, the nut 28 applies a pressure against the top surface 30 of the annular flange 22; the annular flange 22 in turn applies pressure to the washer 26 at the point where the bottom surface 32 of the annular flange 22 contacts the washer 26; the washer 26 then exerts pressure on the curved arm 16 at locations 62, 64 defined by cutouts 82, 84 on the bottom portion of the washer 26.

The spinal cross-connector 100 described herein provides varying spatial arrangements to accommodate the needs of physicians in attaching the spinal fixation rods 1, 2 to the vertebrae of the spine and connecting the spinal fixation rods 1, 2 to each other via the spinal cross-connector 100. The spinal cross-connector 100 provides for three degrees of adjustment which can be used singularly or in combination. When the nut 28 is loosened, the spinal cross-connector provides, first, rotation/twisting of the longitudinal member 12 relative to longitudinal member 18 around a longitudinal axis 76; second, it provides width adjustment along the longitudinal axis 76; and third, it provides rotation of longitudinal member 18 relative to longitudinal member 12 around a vertical axis 78.

Referring again to FIG. 4, the spinal cross-connector 100 allows rotation of the first longitudinal member 12 around the longitudinal axis 76. When the first longitudinal member 12 is rotated around this longitudinal axis 76, the curved arm 16 rotates within the bore 8 of the coupler 4. This rotation around the longitudinal axis 76 allows the spinal cross-connector to accommodate the spatial arrangement of the spinal fixation rods 1, 2 in which, when attached to the vertebrae of the spine, the spinal fixation rods 1, 2 have slightly different orientations with respect to the coronal plane. To accommodate the differences in coronal orientation, the first longitudinal member 12 may be rotated around the longitudinal axis 76 and attached to the spinal fixation rod 1.

The spinal cross-connector 100 further provides adjustment of the longitudinal width of the connector 100, thus accommodating varying distances between the spinal fixation rods 1, 2 when they are attached to the vertebrae of the spine. When the nut 28 is loosened, the pressure exerted by the washer 26 on the curved arm 16 is reduced or eliminated and the curved arm 16 of the first longitudinal member 12 can slide back and forth within the bore 8 of the coupler 4. The spinal cross-connector 100 can be lengthened to the point where the pin 36 near the distal end 34 of the curved arm 16 abuts, and is blocked by, the second side 44 of the head portion 6 of the coupler 4. Similarly, the width of the spinal cross-connector 100 can be reduced by sliding the curved arm 16 of the first longitudinal member 12 through the bore 8 of the coupler 4, to the point that the inside edge 17 of the first longitudinal member 12 abuts the washer 26 or the first side 42 of the head portion 6 of the coupler 4.

The spinal cross-connector 100 also provides rotation of the second longitudinal member 18 longitudinally around the vertical axis 78. This longitudinal rotation of the second longitudinal member 18 around the vertical axis 78 is accomplished by the positioning of the bore 24 of the annular flange 22 of the second longitudinal member 18 over the threaded portion 10 of the coupler 4, thus making the second longitudinal member radially rotatable around the threaded portion 10 of the coupler 4. This longitudinal rotation around the vertical axis 78 allows the spinal cross-connector to accommodate a spatial arrangement of the spinal fixation rods 1, 2 in which, when attached to the vertebrae of the spine, the spinal fixation rods 1, 2 have different orientations with respect to the sagittal plane. For example, in looking at FIG. 4, when the spinal fixation rods 1, 2 are attached to the spine, the far ends (into the page in FIG. 4) of the spinal fixation rods 1, 2 may be closer to each other than the near ends (out of the page).

After the longitudinal members 12, 18 are adjusted to fit the spatial arrangement of the spinal fixation rods 1, 2, the set screws 38, 40 are tightened to secure the spinal cross connector 100 to the spinal fixation rods 1, 2. Then, tightening the nut 28 onto the threaded portion 10 of the coupler 4 acts to secure the spatial arrangement of the longitudinal members 12, 18. Upon securely tightening the nut 28, the first longitudinal member 12 is no longer able to slide longitudinally through the bore 8 or rotate around the longitudinal axis 76, nor is the second longitudinal member 18 able to rotate around the vertical axis 78.

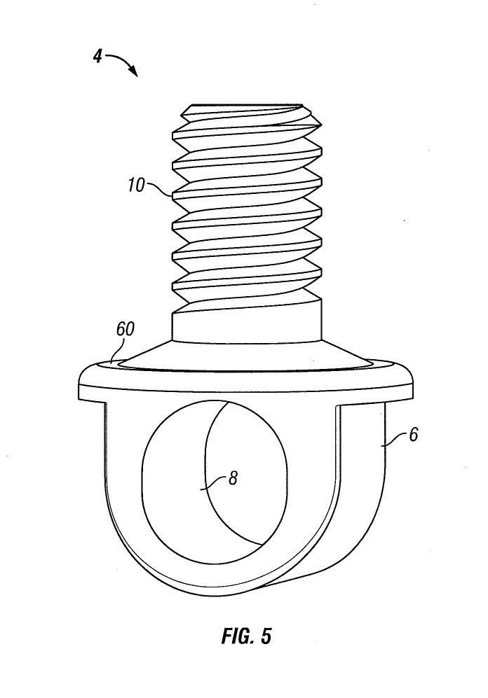

FIG. 5 shows a view of the coupler 4 alone, including the threaded portion 10 and the head portion 6, as well as the top of the head portion 60 and the oblong bore 8 through the head portion 6.

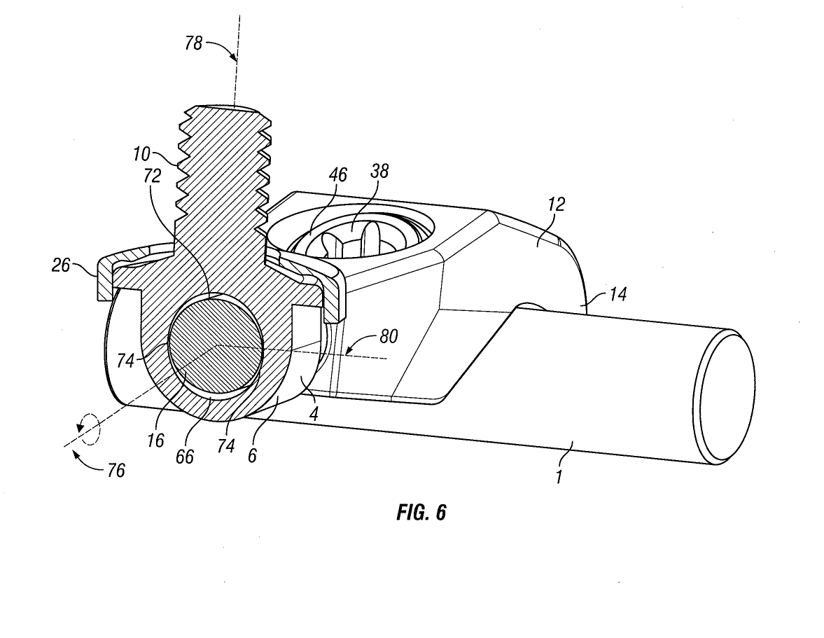

FIG. 6 provides a cross-sectional view of several components of the spinal cross-connector 100, the view being cut along the vertical axis 78, showing the curved arm 16 passing through the middle of the bore 8 of the coupler 4. The rotation of the first longitudinal member 12 within the bore 8 of the coupler 4 is possible, despite the curvature of the curved arm 16, because the bore 8 is oblong shaped. The bore 8 has a sufficiently large vertical diameter such that the curvature of the curved arm 16 is accommodated in the oblong-shaped bore. Because the curved arm 16 is curved, when the curved arm 16 is inserted in the oblong bore 8, the curved arm 16 and the bore 8 make contact at two contact locations 68, 70 (seen in FIG. 4) along the bottom of the bore 8 on both sides of the bore 8. Also as a consequence of the curved arm 16 being curved, a lower gap 66 exists beneath the curved arm 16 between the contact points 68, 70 on the bottom of the bore 8. The curved arm 16 does not make contact with the bore 8 in the lower gap 66.

Furthermore, the curved arm 16 also does not contact the bore 8 at all along the top of the bore 8, which allows for the rotational freedom of the curved arm 16 around the longitudinal axis 76. The space between the curved arm 16 and the upper part of the bore 8 is defined by an upper gap 72. There also exist gaps 74 on both sides of the curved arm 16 between the bore 8 and the curved arm 16. The existence of the gaps 74 on the sides provides additional space for the curved arm 16 to rotate within the bore 8 around the longitudinal axis 76. The curved arm 16 can be a circular diameter, while the bore 8 is oblong shaped, thus the lower and upper gaps 66, 72 can be larger than the side gaps 74.

In one suitable implementation, for example, the vertical diameter of the bore 8 can be 4.51 mm and the horizontal diameter can be 4 mm. The circular cross-sectional diameter of the curved arm 16 can be 3.9 mm. Thus, the lower and upper gaps 66, 72, in this example, are each roughly 0.3 mm and the side gaps 74 are each 0.05 mm wide. The radius of curvature of the curved arm 16 can be, for example, 38.1 mm. The adjustability features of the cross connector are facilitated by a bore 8 that is larger than the curved arm 16, but the bore 8 can be only slightly larger, as the rotation around axis 76 does not need to be large to accommodate the variations is spinal rod coronal orientation encountered in spinal surgeries. A rotation of ±10 degrees is typically sufficient. If desired, more rotational freedom can be provided by increasing the size of the bore 8 relative to the size of the arm 16.

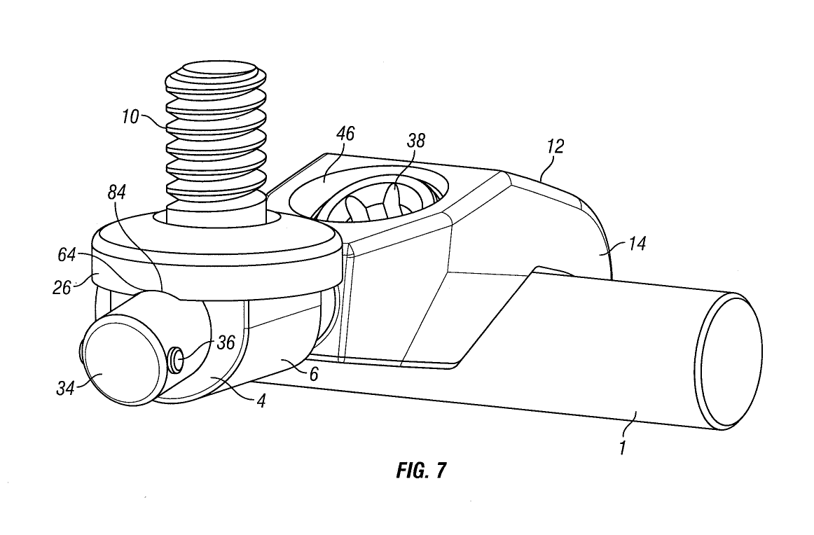

FIG. 7 provides a view of multiple components of the spinal cross-connector 100, including the coupler 4, the washer 26, first longitudinal member 12, and the set screw 38. Visible from this view is the cut out 84 on the washer 26 (an identical cut out 82 is on the other side of the washer 26, visible in FIG. 1). The cut out 84 comprises an arced portion of the washer 26 where the washer 26 makes contact with the curved arm 16 at contact location 64 (similarly, the cut out 82 makes contact with a the curved arm at a contact location 62 on the other side of the washer 26, not visible in FIG. 7). When the nut 28 is tightened on the threaded portion 10 of the coupler 4, the washer 26 applies pressure to the curved arm 16 through cut outs 82, 84 at the contact locations 62, 64, in order to hold the first longitudinal member 12 in place.

When the nut 28 is loosened, the washer 26 applies less pressure (e.g., no pressure) through the cut outs 82, 84 to the curved arm 16, allowing the orientation of the longitudinal members 12, 18 to be adjusted. When a new orientation is established, the nut 28 may be tightened, thus again applying pressure to the washer 26 to hold the longitudinal members 12, 18 in place. Consequently, while the spinal cross-connector can rotate around the longitudinal and vertical axes 76, 78 and move laterally along the longitudinal axis 76 through the coupler 4, the spatial arrangement of the component parts of the spinal cross-connector 100 in relation to each other can be maintained by tightening the nut 28 on the coupler 4.

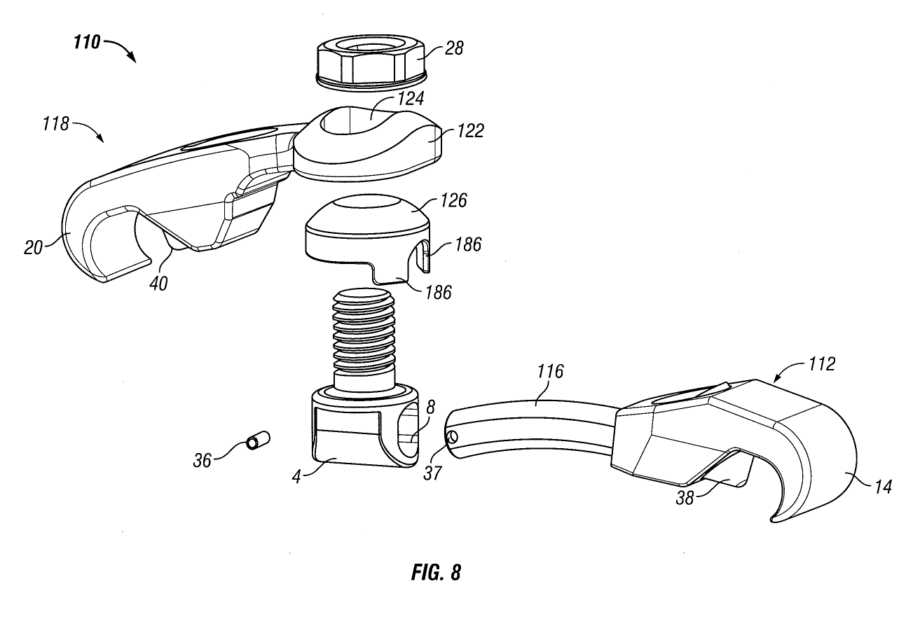

As shown in FIG. 8, another implementation of the spinal cross-connector 110 comprises eight components: a first longitudinal member 112, a second longitudinal member 118, a coupler 4, a washer 126, a pin 36, and a nut 28. Housed within the first and second longitudinal members 112, 118 are respective set screws 38, 40. The washer 126 may be provided with skirt portions 186 on one side of the washer 126. The components of the spinal cross-connector 110 may be made of medical-grade stainless steel, titanium, or other such material suitable for placement in the human body.

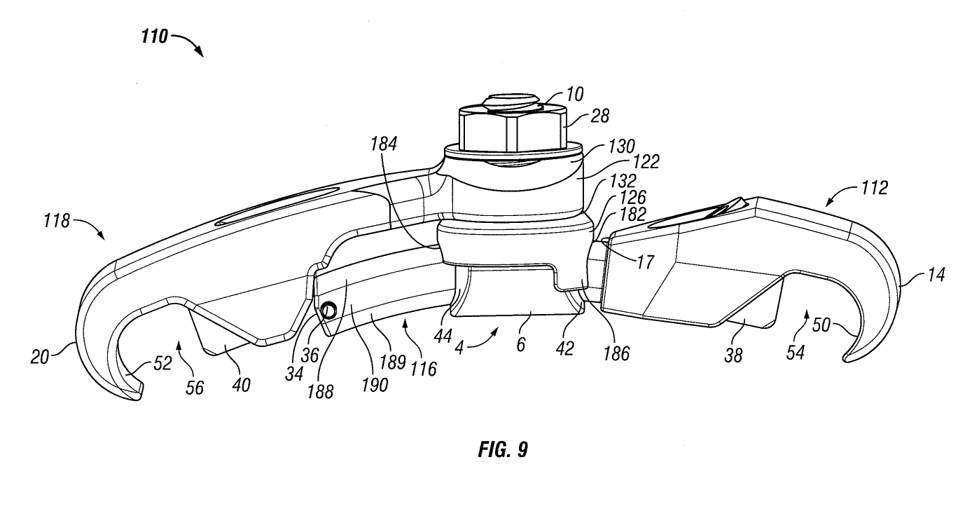

FIG. 9 is a side view of an embodiment of the assembled spinal cross-connector 110. The first longitudinal member 112 comprises a spinal fixation rod coupler 14 on a first end and a curved arm 116 on a second end. When the spinal cross-connector 110 is assembled, the curved arm 116 extends through the bore 8 in the coupler 4 from a first side 42 of the coupler 4 and exits the bore 8 from a second side 44 of the coupler 4. The curved arm 116 has a rounded top 188, a rounded bottom 189, and flat sides 190. The first longitudinal member 112 can slide longitudinally through the bore 8 in the coupler 4 to adjust the width of the spinal cross-connector 110. The spinal cross-connector 110 is longitudinally adjustable to vary the width of the spinal cross-connector 110 according to an individual's anatomy and the distance between the spinal fixation rods.

The washer 126 can include two skirt portions 186 forming a cut out 182 on one side of the washer 126. The washer can include a cut out 184 on the opposite side of the washer 126. In the embodiment shown, when the washer 126 is placed over the coupler 4, the washer 126 is positioned such that the skirt portions 186 of the washer 126 are placed on the side closest to the inside edge 17 of the first longitudinal member 112 and the cut out 184 is placed on the side closest to the distal end 34 of the curved arm 116. In other embodiments, it is contemplated that the skirt portions 186 are placed on the side closest to distal end 34 of the curved arm 116 and the cut out 184 of the washer 126 is placed on the side closest to the inside edge 17 of the first longitudinal member 112.

The top of cut out 182 and the cut out 184 of the washer 126 are shaped to accommodate the rounded top 188 of the curved arm 116. The skirt portions 186 of the washer 126 are shaped to accommodate the flat sides 190 of the curved arm 116. When the nut 28 is tightened on the threaded portion 10 of the coupler 4, pressure is applied through the second longitudinal member 118 to the washer 126. The washer 126 can then exert pressure on the curved arm 116 of the first longitudinal member 112 through the cut outs 182 and 184 and through the skirt portions along the flat sides 190 when the nut 28 is tightened.

The second longitudinal member 118 comprises a spinal fixation rod coupler 20 on a first end and an annular flange 122 on a second end. The annular flange 122 comprises a bore 124 therethrough, which, when the spinal cross-connector 110 is assembled, is positioned around the threaded portion 10 of the coupler 4. The second longitudinal member 118 can rotate on the threaded portion 10 of the coupler 4 around a vertical axis. In the embodiment shown, the bore 124 through the annular flange 122 has an oblong shape. Because of the oblong shape of the bore 124, in addition to rotation around the vertical axis, the second longitudinal member 118 can also slide back and forth relative to the threaded portion 10 of the coupler 4. In other embodiments, it is contemplated that the bore 124 can instead have an enlarged circular bore (substantially larger than the diameter of the threaded portion 10 of the coupler 4) to allow for sliding of the second longitudinal member 118 in more than one direction.

The oblong shape of the bore 124 allows the second longitudinal member 118 to move from side to side relative to the threaded portion 10 of the coupler 4 and also relative to the first longitudinal member 112. The top of the washer 126 is dome shaped. The bottom surface 132 of the annular flange 126 is shaped in a concave manner to fit over the dome of the washer 126. Thus, when the second longitudinal member 118 is adjusted side-to-side relative to the coupler 4, the bottom surface 132 of the annular flange 126 slides over the domed portion of the washer 126. The second longitudinal member 118, in effect, rotates around the longitudinal axis.

In one suitable implementation, the width of the bore 124 in the annular flange 122 can be 4.5 mm and the length of the bore 124 in the annular flange can be 13.7 mm. The circular diameter of the threaded portion 10 of the coupler 4 can be 4.0 mm. In this implementation, the second longitudinal member 118 can experience rotation of ±15 degrees from the nominal center position.

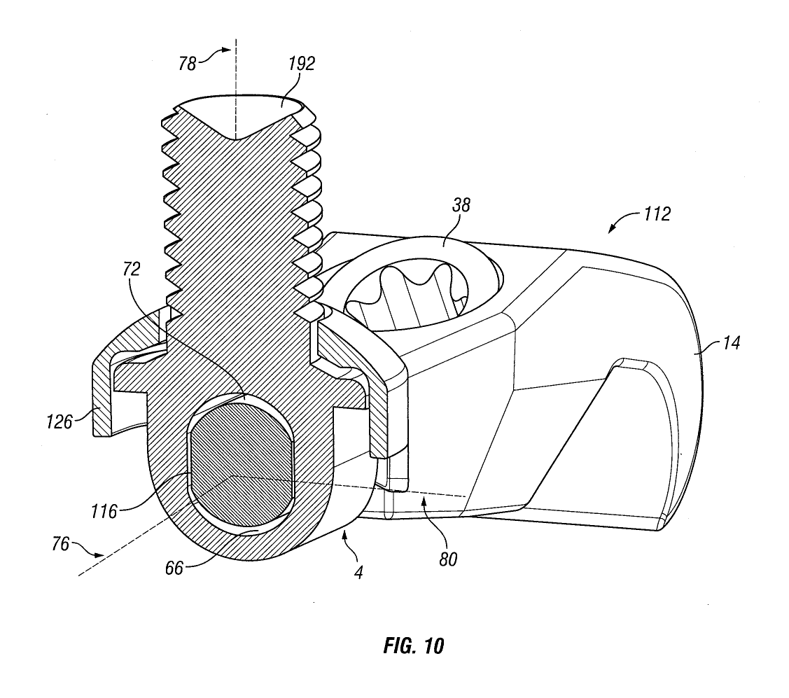

The top surface 130 of the annular flange 122 is spherically contoured, the slope leading downwards away from the bore 124. The sloped nature of the top surface 130 of the annular flange 122 allows for the nut 28 to still fit above the annular flange and be tightened on the annular flange 122 at various angles of the annular flange 122 relative to the threaded portion 10 of the coupler 4. FIG. 10 provides a cross-sectional view of several components of the spinal cross-connector 110, the view being cut along the vertical axis 78, showing the curved arm 16 passing through the middle of the bore 8 of the coupler 4. As a consequence of the curved arm 116 being curved, a lower gap 66 exists beneath the curved arm 116 on the bottom of the bore 8. The curved arm 116 does not make contact with the bore 8 in the lower gap 66. The space between the curved arm 116 and the upper part of the bore 8 is defined by an upper gap 72. There also exist gaps 74 on both sides of the curved aim 116 between the bore 8 and the curved arm 116. The flat sides 190 along with the rounded top 188 and rounded bottom 189 give the curved arm 116 an oblong shape. In this embodiment, the curved arm 116, being oblong shaped, with the flat sides 190 adjacent to the flat sides of the bore 8, exhibits very limited rotational motion when positioned in the oblong bore 8 of the coupler 4.

In one suitable implementation, the vertical diameter of the bore 8 can be 4.51 mm and the horizontal diameter can be 3.65 mm. The vertical diameter of the curved arm 116 can be 3.9 mm and the horizontal diameter of the curved arm 116 can be 3.45 mm. Thus, the lower and upper gaps 66, 72, in this example, are each roughly 0.3 mm and the side gaps 74 are each 0.1 mm wide. The radius of curvature of the curved arm 16 can be, for example, 38.1 mm.

In one implementation, the threaded portion 10 of the coupler 4 may include a cavity 192 in the top. The cavity 192 may be conical in shape. The existence of the cavity 192 in the threaded portion 10 of the coupler 4 allows an upper section (e.g., the first thread) of the threaded portion 10 to be physically deformed with a punch or press after the nut 28 is threaded on, but before the nut 28 is tightened during installation. During manufacture, the upper section of the threaded portion would be purposely deformed after first placing the nut 28 over the threaded portion 10 of the coupler. Deforming the upper section of the threaded portion 10 in this manner prevents the surgeon from accidentally removing and potentially dropping and losing the nut 28 while adjusting and tightening the nut 28.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention, which is defined by the following claims and their equivalents.

What is claimed is:

1. A spinal cross-connector for attachment to spinal fixation rods, the cross-connector comprising:
a coupler comprising a head portion and a threaded portion, wherein the head portion comprises a bore therethrough and the threaded portion extends from the head portion;
a first longitudinal member comprising a spinal fixation rod coupler on a first end and a curved arm extending from the first end and through the bore of the coupler;
a second longitudinal member comprising a spinal fixation rod coupler on a first end and an annular flange at a second end, wherein the threaded portion of the coupler extends through the annular flange;
a washer positioned over the threaded portion of the coupler between the annular flange of the second longitudinal member and the head portion of the coupler; and
a nut engaged with the threaded portion of the coupler and located above the annular flange of the second longitudinal member.

2. The cross-connector of claim 1, wherein the threaded portion of the coupler extends along an axis perpendicular to the bore of the coupler.

3. The cross-connector of claim 1, wherein the bore in the head portion of the coupler is oblong in vertical cross section.

4. The cross-connector of claim 1, wherein the curved arm of the first longitudinal member contacts the bore at only two contact locations.

5. The cross-connector of claim 4, wherein the washer contacts portions of the curved arm at two locations opposite to the contact locations between the bore and the curved arm.

6. The cross-connector of claim 1, wherein the curved arm of the first longitudinal member comprises two straight sides and a rounded top and a rounded bottom.

7. The cross-connector of claim 6, wherein the washer comprises two skirt portions.

8. The cross-connector of claim 7, wherein the skirt portions are configured to fit against the straight sides of the curved arm of the first longitudinal member.

9. The cross-connector claim 1, wherein the nut is tightened against a top surface of the annular flange and the washer is forced against the top portion of the curved arm.

10. The cross-connector of claim 1, further comprising a pin extending through a distal bore in the distal end of the curved arm, the distal bore running in a direction transverse to the bore in the coupler, wherein the pin is of a length greater than the smallest cross-sectional diameter of the bore in the coupler.

11. The cross-connector of claim 1, further comprising two set screws, each set screw extending through a threaded bore in the first and second longitudinal members.

12. The cross-connector of claim 11, wherein each of the screws engages a spinal fixation rod and secures the spinal fixation rod to the first and second longitudinal members.

13. The cross-connector of claim 1, wherein the curved arm of the first longitudinal member is configured to move longitudinally through the bore in the coupler.

14. The cross-connector of claim 1, wherein the curved arm of the first longitudinal member is rotatable inside the bore of the coupler.

15. The cross-connector of claim 1, wherein the annular flange of the second longitudinal member further comprises a bore therethrough and is moveable with respect to the threaded portion of the coupler.

16. The cross-connector of claim 15, wherein the bore of the annular flange is circular and the second longitudinal member is rotatable around a vertical axis.

17. The cross-connector of claim 15, wherein the bore of the annular flange is oblong and the second longitudinal member is rotatable around a vertical axis and slideable in at least one direction.

18. The cross-connector of claim 16, wherein the second longitudinal member is rotatable around the vertical axis and slideable in more than one direction.

19. A spinal cross-connector for attachment to spinal fixation rods, the cross-connector comprising:
a first curved longitudinal member;
a second longitudinal member;
a coupler, coupling the first and second longitudinal members, wherein the coupler comprises an oblong bore configured to accommodate the curve of the first longitudinal member;
wherein the first longitudinal member is rotatable around a longitudinal axis and is configured to move along the longitudinal axis through the coupler; and
wherein the second longitudinal member is rotatable around a vertical axis.

20. The cross-connector of claim 19, wherein the first longitudinal member contacts the coupler at only two contact locations.

21. The cross-connector of claim 19, wherein the first longitudinal member comprises a curved arm.

22. The cross-connector of claim 19, wherein the first and second longitudinal members each comprise a spinal fixation rod coupler.

23. The cross-connector of claim 19, comprising two set screws, each set screw extending through a bore in the first and second longitudinal members.

24. The cross-connector of claim 19, wherein the coupler comprises a head portion, the head portion comprising the oblong bore therethrough and a threaded portion extending from the head portion.

25. The cross-connector of claim 24, further comprising a washer positioned around the threaded portion of the coupler and disposed between the second longitudinal member and the head portion of the coupler.

26. The cross-connector of claim 25, wherein the washer contacts the first longitudinal member at only two contact locations.

27. The cross-connector of claim 19, further comprising a nut engaged with the coupler for securing the first and second longitudinal members to the coupler.

28. A spinal cross-connector for attachment to spinal fixation rods, the cross-connector comprising:
a first curved longitudinal member;
a second longitudinal member;
a coupler, coupling the first and second longitudinal members, wherein the coupler comprises an oblong bore configured to accommodate the curve of the first longitudinal member;
wherein the first longitudinal member is configured to move along a longitudinal axis through the coupler; and
wherein the second longitudinal member is rotatable around a longitudinal and a vertical axis.

29. The cross-connector of claim 28, wherein the first longitudinal member contacts the coupler at only two contact locations.

30. The cross-connector of claim 28, wherein the first longitudinal member comprises a curved arm.

31. The cross-connector of claim 28, wherein the first and second longitudinal members each comprise a spinal fixation rod coupler.

32. The cross-connector of claim 28, comprising two set screws, each set screw extending through a bore in the first and second longitudinal members.

33. The cross-connector of claim 28, wherein the coupler comprises a head portion, the head portion comprising the oblong bore therethrough and a threaded portion extending from the head portion.

34. The cross-connector of claim 33, further comprising a washer positioned around the threaded portion of the coupler and disposed between the second longitudinal member and the head portion of the coupler.

35. The cross-connector of claim 34, wherein the curved arm of the first longitudinal member comprises two straight sides and a rounded top and a rounded bottom.

36. The cross-connector of claim 35, wherein the washer comprises two skirt portions, the skirt portions being configured to fit against the straight sides of the curved arm of the first longitudinal member.

37. The cross-connector of claim 28, further comprising a nut engaged with the coupler for securing the first and second longitudinal members to the coupler.

38. A spinal cross-connector for attachment to spinal fixation rods, the cross-connector comprising:
a first longitudinal member;
a second longitudinal member;
means for coupling the first and second longitudinal members, wherein the coupling means allows the first longitudinal member to move laterally along the longitudinal axis through the coupling means and allows the first and second longitudinal member to rotate relative to one another around a vertical axis and to rotate relatively around a longitudinal axis.

39. The cross-connector of claim 38, wherein the means for coupling is a coupler comprising a head portion and a threaded portion, wherein the head portion comprises a bore therethrough and the threaded portion extends from the head portion.
